(19) United States
(12) Patent Application Publication
(10) Pub. No.: US 2024/0183081 A1
(43) Pub. Date: Jun. 6, 2024

(54) METHOD FOR OBTAINING A THREAD IN ORDER TO PRODUCE DEVICES TO BE IMPLANTED INTO THE HUMAN BODY

(71) Applicant: TECRES S.p.A., Sommacampagna (Verona) (IT)

(72) Inventors: Giovanni FACCIOLI, Monzambano (MN) (IT); Renzo SOFFIATTI, Nogara (VR) (IT)

(21) Appl. No.: 18/441,730

(22) Filed: Feb. 14, 2024

Related U.S. Application Data
(63) Continuation-in-part of application No. 18/243,439, filed on Sep. 7, 2023.
Application No. 18/243,439 is a continuation-in-part of application No. 15/551,497, filed on Aug. 16, 2017, now abandoned.

(30) Foreign Application Priority Data
Feb. 16, 2015 (IT) VR2015A000022

(51) Int. Cl. D01D 5/08; A61L 27/16; A61L 27/54; B33Y 10/00; B33Y 70/10

(52) U.S. Cl. CPC D01D 5/082; A61L 27/16; A61L 27/54; B33Y 10/00; B33Y 70/10; A61L 2300/104; A61L 2300/232; A61L 2300/25; A61L 2300/406; D10B 2321/0211; D10B 2401/13; D10B 2509/00

(57) ABSTRACT

Method for the obtainment of a thread made of a material biocompatible and implantable in the human body: a thread for the obtainment of a device implantable in the human body.

### TECHNICAL FIELD OF THE INVENTION

The present invention concerns a biocompatible and implantable material to be used to mould devices able to be implanted in the human body such as catheters, screws or joints, couplings or meshes, spacer devices, prostheses or prosthetic devices, etc. Furthermore, the present invention concerns a method for producing such material and for producing such devices with a 3D printing machine or through injection or compression moulding.

### STATE OF THE ART

The technology of additive production or three-dimensional printing of objects is currently becoming increasingly popular.

In particular, three-dimensional or 3D printing, which represents a natural evolution of 2D printing, allows an object to be reproduced from a three-dimensional model through the superimposition of successive layers of material, until the desired shape is obtained. Such a three-dimensional model is obtained through suitable software.

3D printers are generally faster, more reliable and simpler to use with respect to other technologies for additive production and, moreover, offer the possibility of moulding and assembling parts made up of different materials with different physical and mechanical properties in a single construction process.

The operation of a three-dimensional printer is based on the use of a 3D file developed by suitable software.

The 3D model of the object of interest is prepared—through the aforementioned software—in a series of portions or layers in cross section thereof.

Each portion or layer is then printed one onto the other, in order to recreate the entire three-dimensional object.

There are different 3D printing technologies; their main differences concern, among others, the way in which the various layers are printed.

Some methods, in order to produce the various layers, use materials that melt or soften. Some examples of such technology are “Selective Laser Sintering” (SLS) or “Direct Metal Laser Sintering” (DMLS) or “Fused Deposition Modeling” (FDM).

Other methods provide for the deposition layer-by-layer of liquid materials, which are made to set with different technologies. An example of such a technology is known as “Digital Light Processing” (DLP).

Furthermore, in the case of lamination systems there are thin layers that are cut according to the desired shape and then joined together through known techniques.

A first 3D printing method consists of a printing system by extrusion of material. The printer creates the model one layer at a time, spreading a layer of powder (plaster or resins) and distributing a binder thereupon, for example printing like with an inkjet.

The process is repeated layer by layer until the desired shape of the object is obtained.

In this way, it is possible to make projections in the finished product.

FDM technology, used in conventional quick prototyping, provides for a nozzle adapted for depositing a molten polymer layer-by-layer on a support structure.

Furthermore, some 3D printers for additive synthesis use a thread of thermoplastic polymer as construction material.

In the field of orthopaedics, three-dimensional printing technology has led to bone or parts thereof being made. For example, it has been possible to implant a cranium printed in 3D to a patient.

The skullcap was made with a special resin through the use of a 3D printer. Other recently developed prostheses were obtained through three-dimensional printing of a titanium-based material.

The materials usually used for 3D printing are: plastic materials, for example thermoplastic polymers (for example for SLS and FDM), metals, sand, glass (for example for SLS), photopolymers (for example for stereolithography), laminated sheets (often of the paper type) and relative glues, titanium alloys (for example for “Electron beam melting” or EBM), resins, clays, ceramic, etc.

In particular, for jet or thread 3D printing, the material used must be melted and extrudable through a nozzle suitable for the purpose.

Therefore, one of the problems to be solved in order to be able to three-dimensionally print some objects, particularly for medical or orthopaedic use, is precisely that of making materials having the appropriate characteristics for the final purpose for which the object must be made and at the same time characteristics suitable for the selected three-dimensional printing method.

Basically, surgical practice provides for implanting resins or plastic materials in the body for a short time (for example for catheters) or permanently (for example for polymethyl methacrylate or PMMA, constituent of bone cement, for ultra high molecular weight polyethylene or UHMWPE or in short PE, as friction surface in hip, knee, shoulder prostheses etc., or for polyetheretherketone or PEEK, as cranial prostheses, spinal cages, etc.).

Except for the PMMA of bone cement, all of the other plastic materials undergo mechanical processing in order to obtain a prosthesis or a device to be implanted in the human body.

This occurs both due to objective difficulty in moulding those specific plastic materials, and particularly for the extremely heterogeneous demand of prostheses or devices to be implanted in the human body of greatly different shape and size, requirements which only mechanical processing can quickly satisfy.

However, these plastic materials (for example PE and PEEK) do not and cannot contain pharmaceutical or medical substances, like for example one or more antibiotics, nor radiopacifying agents, like for example barium, nor possible further medical additives used to treat the patient or for surgical treatment.

Such substances, however, would be very useful and appreciated inside such plastics in order to carry out a healing function (for example through local antibiotic/s) and be easily detected radiographically, as done on the other hand by antibiotic-loaded bone cement provided with radiopacifying agents.

Therefore, there is a need for a biocompatible and implantable material, added with pharmaceutical or medical substances and/or radiopacifying agents and/or other useful additives, to be used for moulding, also three-dimensional printing moulding, of devices to be implanted in the human body or spacer devices.

The application WO2012/007535 discloses a part for endosseous implantation molded through injection moulding of a material comprising a thermoplastic binder (preferably PEEK) that incorporate fibers; TCP and zeolite can be incorporated into the binder, the latter substance being able to confer radiopacity at the implant.

The application EP0472237 discloses a material comprising UHMWPE and inorganic filler such as calcium phosphate or hydroxyapatite.

The application DE102007052519 discloses a medical implant, comprising a polymeric material (such as polyethylene or polypropylene) and an antimicrobial composition (including silicon dioxide and metal-containing nanoparticles).

The application WO2010/096053 discloses a medical implant incorporating a medical substance, such as silver or penicillin.

The application WO2013/184010 discloses a middle ear prosthesis including silver powder.

The U.S. Pat. No. 6,641,831 discloses a non-degradable medical product comprising at least two substances: substance A and substance B, wherein substance A is more lipophilic than substance B and has a given solubility in water, lower than that of substance B. At least one, among substance A and substance B, is a pharmaceutically active agent, i.e. an antimicrobial substance.

### BRIEF DESCRIPTION OF THE DRAWINGS

Other features and advantages of the invention will be more evident from the description of an embodiment of a material according to the present invention, illustrated by way of example in the accompanying drawings in which:

FIG. 1 shows an extruder for a material according to the present invention,

FIG. 2 shows a granulated or pellet plastic material,

FIG. 3 shown an enlarged version of a thread obtained by an extruder according to the present invention, and

FIG. 4 depicts a flow chart showing a method of obtainment of a thread according to an aspect of the present invention.

In the accompanying drawings, identical parts or components are identified by the same reference numbers.

### SUMMARY OF THE INVENTION

The task of the present invention is to improve the state of the art.

In such a technical task, an aim of the present invention is to provide a biocompatible and implantable material, adapted for being used in moulding technology, possibly also three-dimensional printing, and which can be added to with pharmaceutical or medical substances and/or with further additives. An aim of the present invention is to provide a biocompatible and implantable material that is extrudable.

In accordance with an aspect of the present invention, a method for obtaining a biocompatible and implantable material is provided according to the present application.

In accordance with another aspect of the present invention, a temporary and/or disposable device to be implanted in the human body or a spacer device for the treatment of a bone or joint location made with a biocompatible and implantable material is provided, according to the present application.

In accordance with another aspect of the present invention, a biocompatible and implantable material is provided according to the present application.

An advantage of such a device to be implanted in the human body or a spacer device for the treatment of a bone or joint seat consists of being able to be added to with pharmaceutical or medical substances and/or with additives and be made through moulding, possibly three-dimensional moulding.

A further advantage of the device to be implanted in the human body or a spacer device for the treatment of a bone or joint location consists of being able to be personalised or made in series in a quick and simple manner, substantially without the need for further surface finishing processing.

In accordance with another aspect of the present invention a method for obtaining a biocompatible and implantable material is provided, adapted for being used in printing technology, possibly also three-dimensional printing, and which can be added to with pharmaceutical or medical substances and/or with a radiopacifying agent and/or with further additives, according to the present application.

An advantage of such a method is that it is quick and simple, substantially without the need for further surface finishing processing steps.

The present application refers to preferred and advantageous embodiments of the invention.

### DETAILED DESCRIPTION OF THE INVENTION

The present invention refers to a material biocompatible and implantable in the human body, for the obtainment of a device to be implanted in the human body or a spacer device for the treatment of a bone or joint seat. Specifically, the device of the present invention is temporary and/or disposable.

Such a material can comprise one or more of the following materials: an acrylic resin or a plastic material or polyethylene (PE) or low density polyethylene or high density polyethylene or low molecular weight polyethylene pr medium molecular weight polyethylene or ultra high molecular weight polyethylene (UHMWPE) or polypropylene or polyamide or polyetheretherketone (PEEK), or a mixture thereof or a plastic material selected to have a melting point lower than the degradation temperature of at least one antibiotic.

As far as PE is concerned, it is a polymer of several kinds, most of which having the chemical formula (C₂H₄)n. PE is usually a mixture of similar polymers of ethylene, with various values of n. It can be low-density (LDPE) or high-density (HDPE) and many variations thereof. Its properties can be modified further by crosslinking or copolymerization.

LDPE is softer and more transparent than HDPE. For medium- and high-density polyethylene the melting point is typically in the range 120 to 130° C. while the melting point for average commercial low-density polyethylene is typically 105 to 115° C.

However, for the reasons explained in the present specification, the melting point of the melting temperature of PE does not directly correspond to the moulding temperature.

For example, known moulding or operating temperatures of LDPE and HDPE are in the range of 180-240° C. and 200-260° C. respectively, notwithstanding the above indicated melting points.

Such plastic materials, in a version of the invention, are insoluble.

In a further version of the invention, the plastic material is soluble and comprises, for example, polylactic or polyglycolic acid polymers or other suitable polymers.

Possible acrylic resins include an acrylic copolymer made up of MMA, styrene and ethyl-acrylate or polymethyl methacrylate or mixtures comprising acrylic polymers and/or copolymers.

One of the main characteristics of such a material is that it is mouldable, for example through injection moulding or through three-dimensional printing or through forming presses or through thermoplastic moulding technology.

In this way, especially when it concerns three-dimensional printing, it is possible to mould such a material according to a simple and quick procedure, capable of providing both medical devices or spacer devices produced in series, and personalised devices. For example, the molding step may comprise feeding at least one thread of such material to a tridimensional printer in order to obtain the device according to the present invention.

In the latter case, indeed, it is possible to obtain a three-dimensional model of the device to be obtained, by selecting the size and shape or configuration most suitable for the surgical or anatomical requirements of the patient, and produce the relative device adapted to the specific patient.

In the aforementioned cases, however, thanks to the mouldability of the material according to the present invention, it is possible to obtain finished devices, substantially without the need to carry out further surface finishing or processing steps thereof.

The material according to the present invention also comprises at least one additive such as a pharmaceutical or medical substance and/or a radiopacifying agent and/or a further additive. The pharmaceutical or medical substance comprised in the material according to the present invention can comprise or consist of at least one antibiotic, for example gentamicin sulphate or another suitable antibiotic, such as one of more of the following: Amikacin, Azlocillin, Aztreonam, Clarithromycin, Chloramphenicol, Ciprofloxacin, Clindamycin, Coumermycin, Fosfomycin, Josamycin, Kanamycin, Mezlocillin, Mupirocin, Nalidixic acid, Netilmicin, Norfloxacin, Novobiocin, Ofloxacin, Oxacillin, Sulbactam, Tobramycin, Trimethoprim, Trimethoprim+Sulphamethoxazole, Vancomycin, another suitable antibiotic which is thermoresistant and that is active against microbial infections, etc.

As far as the indicated antibiotics, they have a known melting point generally considered at normal conditions (i.e. 1 bar of pressure). For example, the melting point of the gentamicin sulphate is between 218-237° ° C. at 1 bar of pressure.

However, according to the present invention, it is not possible to take into account such known melting points because, when the antibiotics are added to the plastic material in a press to mould the plastic material mixture, the pressure is variable and can reach some hundreds of bars. In such conditions, the thermal degradation of the antibiotic can take please at really lower temperatures. Therefore, the operative environment behaves differently from theoretical conditions, for degradations of pharmaceutical or medical substances.

In fact, it has been demonstrated by the Applicant that gentamicin sulphate can be at least partially degraded (for example about 10% of degradation) even if moulded at a temperature below its melting/degradation point, because the moulding step occurs under pressure, i.e. at a pressure higher than 1 bar. Such degradation results in a slightly yellow colour in the obtained product, compared to fully white coloured devices obtained with completely active antibiotics.

According to a version of the present invention, the moulding and/or extruding temperature in the present invention is about 160° C. or lower, in order to obtain, thanks to the present invention, fully active antibiotics or pharmaceutical or medical substances.

The pharmaceutical or medical substance comprised in the material according to the present invention can further comprise or consist of at least an antiseptic agent, of organic or inorganic nature, a bacteriostatic agent, like for example silver in its various forms, such as metallic powder or salts comprising citrate, proteinate, colloidal, electrolytic, or other forms that can be used in the human body, boric acid, etcetera.

For example, if the antiseptic agent or the bacteriostatic agent is metallic silver, this is insensitive to the melting temperature of the plastic material that would receive it according to the present invention.

Moreover, variously salified silver, for example colloidal silver powder, is insensitive to the melting temperature only of a few plastic materials of the material according to the present invention.

Possible bacteriostatic agents also comprise copper and/or gold, as well as the aforementioned silver, in their various forms, for example their salts or components. Such materials are, indeed, thermostable. Other thermostable inorganic substances having a medicating action can be advantageously included in the molten plastic material. A further example is boric acid that has an antiseptic action and is thermostable at over 300° C.

Such an option is particularly relevant when the device to be obtained with said material is a spacer device for treating an infection present in a bone or joint location.

The function of spacer devices, indeed, is to maintain the joint space left by an infected prosthesis, which for this reason is removed, and at the same time to treat the infection of the bone location, internally comprising for example a pharmaceutical or medical substance, like for example at least one antibiotic, to be eluted in the area to be treated.

The material according to the present invention, alternatively or in addition, could also comprise a radiopacifying agent, such as for example, metallic powders or heavy metal powers, for example, of tungsten, tantalum, silver or a metal salt of barium sulphate, zirconium oxide, bismuth oxide, etcetera.

According to a variant of the present invention, the radiopacifying agent is bismuth oxide, considered alone or in combination with silver metallic powders or salts. In this way, with silver it is possible to have at the same time radiopacifying and bactericide activities.

With bismuth oxide, it is possible to use a minor quantity of radiopacifying agent because it has a yellow coloration (which is not present in other radiopacifying agents) and therefore it clearly distinguishes materials. Furthermore, the minor quantity of radiopacifying agent is due also to the specific nature of the bismuth oxide material.

Such agents, as known, are visible to X-rays and thus make it possible to monitor the position of the device to be implanted in the human body or the spacer device, as well as the material that contains them according to the present invention.

The material according to the present invention, moreover, can comprise further medical additives, such as for example, soluble and/or reabsorbable ceramic material, in the form of powder or granules, comprising tricalcium phosphate or calcium sulphate or hydroxyapatite, etcetera, or colouring substances of the biocompatible type and adapted to be introduced in the human body, etcetera.

Such additives, if they are not soluble or reabsorbable, can stay permanently inside the human body, or be removed if the biocompatible and implantable material in which they are contained is removed.

One of the problems to be solved, by a material provided according to the present invention, is that the substances contained therein can be degraded or undergo modifications due to the temperatures and/or pressures that a material according to the present invention encounters, namely, when such a material is heated or when it is moulded, for example through injection moulding or three-dimensional printing or another moulding technique.

A material according to embodiments of the present invention can be in the form of a thread, for example, having a diameter that can vary from between 1 and 10 millimetres. preferably from between 1 and 5 millimetres or from between 5 and 10 millimetres. In particularly useful embodiments, the thread diameter can range from between 2 and 5 mm, or exceeding 5 mm to 10 mm, for example, and is adapted for being used to feed a three-dimensional printer of the thread or inkjet type or in another moulding technique. [0064] One of the methods for allowing the material according to the present invention to be reduced to a thread adapted for being extruded and/or injected and/or printed three-dimensionally is as follows.

The base material is provided, for example, in the form of a pellet(s) (e.g. press pellet) or of granules; the material is inserted in suitable machinery, for example, an extruder, and an additive such as at least one from a pharmaceutical or medical substance and/or a radiopacifying agent and/or a further additive is added.

Then the whole mixture is heated until a certain temperature is reached, for example the melting temperature thereof, or to a temperature suitable for melting or softening the material in question (together with the possible mixture of additives that are added), to such a point as to be able to be extruded in threads or mouldable. Sometimes such temperature is higher than the melting temperature, while some other times it is slightly lower than the melting temperature.

Such a melting temperature varies as the polymers comprised according to the present invention varies. Most of such polymers have melting values comprised between 60° C. and 300° ° C. Such a melting temperature can, for example, be around 250° ° C. One important point to be considered is that, while plastic material in general melts at the melting temperature, it is possible, especially for some pharmaceutical or medical substances, preferably antibiotics, that at the melting temperature they undergo degradation. Therefore, at the temperature wherein the plastic melts, the antibiotics lose their properties. This is the reason why the plastic melting temperature has to be lower than the melting/degradation temperature of the included pharmaceutical or medical substance, preferably the at least one antibiotic.

The at least one radiopacifying agent can be included at the time of forming the thread or the granules.

In a version of the invention, there is then a step of extruding the heated or molten material through suitable machinery, for example the same extruder, in the form of one or more threads having the diameter indicated above.

Such at least one thread can be wound in a coil so that, being extruded, it cools down and becomes consolidated and thus is suitable to then be handled and stored.

Such at least one thread can be used to feed a nozzle of an injection mould or a 3D printer.

Such a thread can be moulded through a three-dimensional printer or through injection moulding or through forming presses or through a thermoplastic moulding technique, in order to obtain a device that can be implanted in the human body or a spacer device for treating a bone or a joint location.

Such a method can, additionally or as an alternative to those described above, provide for a step of crushing or granulation of the material, possibly after cooling thereof.

In a version of the invention, such a step is carried out through a suitable granulating machine.

Thereafter, the base material, or the crushed or granulated material, is used in a thermoplastic press with which moulded products are obtained.

In this case, in a version, a thread is not obtained before moulding, whereas in a second version, it is the thread obtained as indicated earlier that is crushed or granulated, possibly after cooling thereof.

The crushed or granulated material is then moulded, for example through an injection moulding press.

As stated, the temperature at which the material is melted must be below the degradation or damaging temperature of the pharmaceutical or medical substance, and/or of the radiopacifying agent and/or of the further additive, so that they maintain their original characteristics and activity also, following melting of the material, in the thread or resulting material.

Therefore, in a version of the invention, the extrusion or the moulding (but not the 3D printing) is carried out at lower temperatures than that at which the aforementioned substances degrade, by suitably selecting plastic materials with particularly low melting or softening temperatures, for example below the melting or degradation temperature of the pharmaceutical or medical substance, in order to obtain the end product comprising at least one from a pharmaceutical or medical substance, or a radiopacifying agent or a further additive, according to the specific requirements.

For example, an antibiotic that has a melting temperature of 180° C., so as not to degrade during moulding, must not exceed such a temperature.

Through the aforementioned material, it is possible to obtain devices that can be implanted in the human body like for example catheters, screws or joints, couplings or meshes, for which it may be useful to have a medicated version, a non-medicated version, an X-ray visible version, a coloured version, etcetera.

Alternatively, through the aforementioned material, it is possible to obtain devices that can be implanted in the human body such as, for example, medical “threads”, for which it may be useful to have a medicated version, for example, comprising an antibiotic and/or a radiopaque substance.

In a version of the invention, the material is adapted for making devices that can be implanted in the human body or spacer devices initially without pharmaceutical or medical substances but capable of absorbing such substances at a later time, after they have been moulded.

In this way and/or thanks to the present invention, if the material in question requires a melting temperature to obtain it in the form of thread or a moulding temperature above the degradation temperature of the pharmaceutical or medical substance, it will equally be possible to obtain the device in question without the risk of damaging them, and insert the substances of interest at a later time, without the risk of damaging them. In this case, the device in question is porous. The porosity of the device makes it capable of absorbing, for example, by capillary action, such substances after it has been formed.

In a further version, such devices are made with a material to which is already added at least one pharmaceutical or medical substance, but, being porous, once formed such material can be capable of absorbing another substance which is the same or different with respect to the substance already contained in them.

In this way, it has been seen how the material according to the present invention, being able to be moulded and/or being able to be made in the form of a thread, allows devices able to be implanted in the human body or spacer devices to be obtained in a quick, simple and if desired, a customizable manner in terms of the shape and the size, and also with regards to the pharmaceutical or medical substances or the further additives or agents desired to be contained therein, according to the surgical and/or anatomical requirements of the patient.

The present invention also refers to a device that can be implanted in the human body, or to a spacer device for treating a bone or a joint location, comprising a material biocompatible and implantable in the human body according to the present invention.

Such devices, in fact, comprise an additive such as a pharmaceutical or medical substance and/or a radiopacifying agent and/or a further additive, as described earlier for the material according to the present invention.

Such devices are made by moulding, for example through a three-dimensional printer or through injection moulding or through forming presses or through a thermoplastic moulding technique.

Another embodiment that can be obtained with the material according to the present invention is a cranial prosthesis. In this way, the prosthesis could be made directly from the CAT data, formed according to the configuration and the dimensions necessary for the anatomical and implanting requirements, and then implanted in the bone location of interest.

Such a cranial prosthesis could contain a pharmaceutical or medical substance and/or a radiopacifying agent and/or a further additive.

As far as the thermal degradation of the active ingredients is concerned, the present invention is successfully able to lower the melting point and/or to improve the flowability and therefore the extrudability of surgically used plastics such as UHMWPE. This ability is surprisingly linked to the feature of the present invention of mixing UHMWPE with a low molecular weight PE and/or with a medium molecular weight PE and/or with a polyethylene having a molecular weight lower than UHMWPE. Therefore, UHMWPE is mixed with at least one PE with a lower molecular weight. In this way, in fact, it is possible to melt such mixture, make it extrudable and reduce it to a thread. In such an obtained thread, if at least one antibiotic is present inside, such antibiotic(s) maintains its active status.

According to an example of the present invention, the plastic mixture comprises or consists of UHMWPE and at least one of a low-density PE and a high-density PE, but always with a low or medium molecular weight or with a molecular weight lower than UHMWPE. Specifically, such plastic mixture comprises or consists of:
UHMWPE and low molecular weight PE, or
UHMWPE and medium molecular weight PE, or
UHMWPE, low molecular weight PE and medium molecular weight PE.

In particular, UHMWPE in the plastic mixture guarantees the mechanical properties of the resulting material, and therefore its percentage typically has to not go below a certain value. However, considering that the present invention is not mainly focused on providing permanent prosthesis, but rather temporary and/or disposable devices, the mechanical performances can be less stringent than for permanent prosthesis.

In a plastic mixture according to the present invention, UHMWPE can be present according to the following percentages: at least 40%, at least 50%, at least 60%, at least 70%, at least 80%, at least 90%, or preferably 40%, 55%, 65%, 75%, 85%, 95%. The higher the percentage of UHMWPE, the higher mechanical properties are present in the resulting material. However, the higher the percentage of UHMWPE, the lower is the flowability of the resulting material. Therefore, the specific percentage depends on the use and/or type of the resulting device.

The remaining percentage of the mixture is made of the at least one of a low or medium molecular weight PE. Preferably, when such components (e.g., both a low or medium molecular weight PE) are present, they are present in an equal amount. Alternatively, it is preferable to have more low molecular weight PE than medium molecular weight PE. Otherwise, it is preferable to have more medium molecular weight PE than low molecular weight PE.

According to a version of the invention, a plastic mixture is provided which comprises 70-80% of UHMWPE and about 20-30% of PE with lower molecular weight, considering that the total percentage refers to the plastic mixture.

In particular, UHMWPE has a relative low melting temperature (135-140° C.), but it has a very low flowability and a high viscosity. In fact, melted UHMWPE is very viscous, and it does not really flow for example, in a mould. In fact, it is usually worked mechanically, for example, with a lathe. Its feature is to have poor flowability.

In more detail, UHMWPE is not mouldable by extrusion nor with a compression press because its molecular weight is so high (about 1 million and more) and its very long chains are not able to slide, not even when they are stretched at high temperatures, such as a temperature higher than its melting point. For these reasons, UHMWPE is produced in plates, bars and tubes, which are then tool machined. On the contrary, according to an aspect of the present invention a mixture with PE having lower molecular weight than UHMWPE is used in order to solve the UHMWPE flowability problem and/or in order to lower its extrusion temperature.

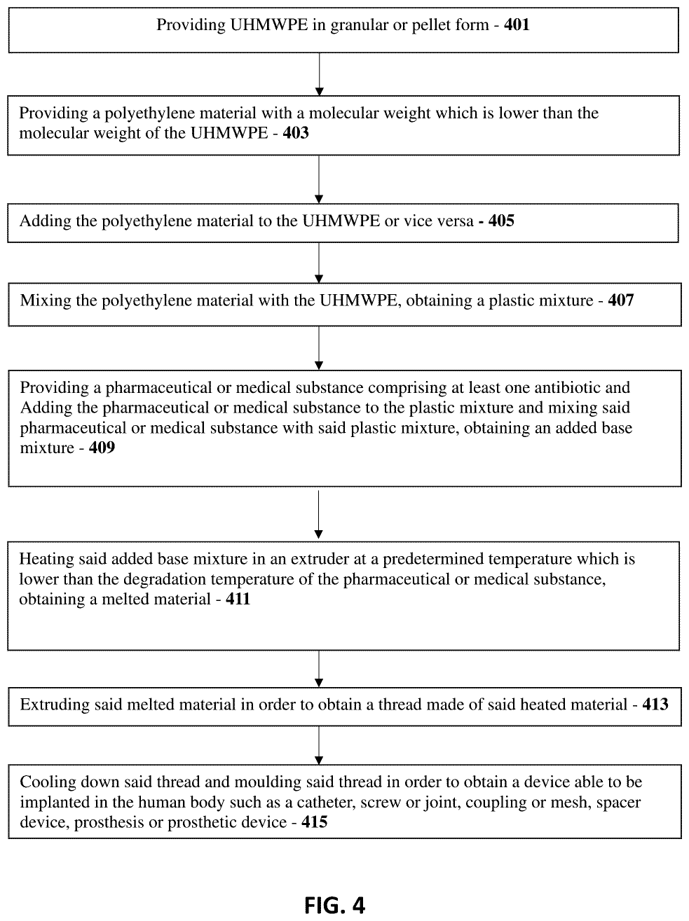

FIG. 4 is a flow diagram depicting exemplary steps of a method for obtaining a thread made of a material biocompatible and implantable in the human body according to an aspect of the present invention.

In one aspect, a method for obtaining the material of the present invention therefore foresees a step of providing UHMWPE, preferably in granular or pellet form (step 401), of providing a polyethylene material with a molecular weight which is lower than the molecular weight of the UHMWPE (step 403), of adding to it, for example, if desired step-by-step or by incremental steps, one or several parts of PE having a lower or intermediate molecular weight to the UHMWPE (step 405) and mixing such PE and UHMWPE together to obtain a plastic mixture (step 407).

With regards to step 405, vice versa is also possible, namely, for example, providing one or several parts of PE with lower or intermediate molecular weight and adding to it—if desired step-by-step or by incremental steps—UHMWPE, preferably in granular or pellet form, to obtain a mixture.

In step 409, at least one additive such as a pharmaceutical or medical substance (e.g., an antibiotic as for example described above) and/or a radiopacifying agent and/or a further additive may be added and mixed to the plastic mixture to obtain an ‘added base mixture’.

In step 411, the added base mixture is heated in an extruder at a predetermined temperature which is lower than a degradation temperature of the added pharmaceutical or medical substance, thus obtaining a melted material.

In step 413, the melted material is extruded in order to obtain a thread made of the melted material.

In step 415, the extruded thread is cooled and moulded in order to obtain a device able to be implanted in the human body, such as e.g., a catheter, a screw or joint, a coupling or mesh, a spacer device, a prosthesis or a prosthetic device.

It is to be noted that such PE according to the present invention can also be called “low density” (LDPE) and/or “high density” (HDPE) polyethylene, which have shorter chains with respect to UHMWPE, but according to an aspect of the present invention, the important point is that LDPE and HPE have low or medium molecular weight in order to reduce the overall molecular weight of the mixture.

Usually, UHMWPE has a molecular weight in the range between 3 million and 6 million, and HDPE has a molecular weight in the range 300.000 to 1 million. When the molecular weight tends to the minor or lower limit, it is a low molecular weight PE while if its molecular weight tends to the upper limit, it will be considered medium molecular weight PE.

According to a version of the invention, the low molecular weight PE can have a molecular weight in the range 200.000 to 500.000.

According to a version of the invention, the medium molecular weight PE can have a molecular weight in the range 500.000 to 1 million.

HDPE has a linear chain, and also the UHMWPE has a linear chain; therefore they are similar. Therefore, mixing them in order to obtain the extrudable mixture according to the present invention guarantees better mechanical performances and/or properties (i.e. there is only a minor deterioration of the mechanical performances, when compared to the mechanical performances of UHMWPE alone).

LDPE, on the contrary, has a branched chain and poor mechanical properties. Therefore, mixing LDPE with UHMWPE determines for the mixture, in at least one version of the invention, poor mechanical properties. Obviously, such mixture can be selected when such mechanical resistance is not important or necessary for the resulting device. Otherwise, it is not the better choice.

A mixed material according to an aspect of the present invention has a reduced molecular weight which allows the sliding of such plastic mixture in the mould or extruder, in order for the same to be moulded, notwithstanding the presence of the poor flowable UHMWPE. With regards to when the PE with lower molecular weight is added step-by-step, this can mean, e.g., that the PE is added time to time at small percentages/amounts at each time, until the desired flowability of the resulting mixture is obtained.

Also, “step-by-step” can mean that the UHMWPE (which is not extrudable) is added time to time, and/or at small subsequent quantities, to the lower molecular weight PE (which is extrudable), until the obtainment of the extrusion of the mixture.

According to another version, it is also possible to add the entire quantity of PE to the UHMWPE, mix them and obtain the desired flowability of the mixture.

In this way, the obtained material has good mechanical properties (not so good as those of pure UHMWPE, but the present invention has the aim to obtain, for example, temporary and disposable devices which have to be maintained in the human body for a limited period of time—only or about 6 months for example—till the healing of the infection). Therefore, an advantageous material according to the present invention has been obtained thanks to the fact that the molecular weight of the mixture has been reduced time to time, a little at a time. The prior art does not specifically disclose the mixture of UHMWPE and other kinds of PE, such as those indicated in the present invention, in order to solve the mouldability problem of UHMWPE.

A mixture of UHMWPE and at least one of a low and/or medium molecular weight PE according to an aspect of the present invention is important to increase the flowability of the plastic material of the present invention. Therefore, the viscosity of a mixture according to the present invention is lower than the viscosity of the UHMWPE alone. In this way, it is easier to extrude (or to create a thread of) the plastic material or the mixture, with respect to the extrusion of the UHMWPE alone.

The melting temperature of a mixture according to the present invention can be affected or not by the presence of at least another PE, in addition to the UHMWPE.

Another advantage, when a material according to the present invention comprises UHMWPE, is that it does not adhere to the bone cement used to cement the device. In this way, when the device is temporary and/or disposable, thanks to the presence of UHMWPE, it is possible to remove the device in an easy way (for example for catheters or spacer devices), because as stated, such plastic material according to the present invention does not stick to the bone cement.

Furthermore, the use of UHMWPE allows, for example, screwing/connecting properties (for example when the device is a screw, joint, etc. to be used in the human body). Furthermore, the resulting material is resistant to breakage, and is flexible.

Another advantage can be conferred by the formation with such material of a mesh, for example for use at a cranial level, in order to “trap” the bone cement inside the openings of the mesh and at the same time providing such mesh made of a flexible and resistant to breakage material, able to trap possible fragments derived from the bone cement.

According to an example of the present invention, the material can comprise:
1) Low and/or medium molecular weight PE: from 5% to 40% or from 5% to 20%
2) Pharmaceutical or medical substance, such as for example, at least one antibiotic, for example, gentamicin sulphate: from 0% to 10% or from 5% to 10%,
3) Another pharmaceutical or medical substance, such as for example, at least another antibiotic, for example vancomycin, for example the hydrochloride salt of vancomycin or vancomycin hydrochloride: from 0% to 10% or from 5% to 10%,
4) Radiopacifying agent: from 0% to 20% or from 10% to 20%,
5) UHMWPE: up to 100%.

As already said, the higher the percentage of UHMWPE, the higher the mechanical performances are of the resulting material. Therefore, the choice of the correct percentages depends on the load and/or mechanical stress which the resulting material/device is subjected to.

According to a preferred version of the invention, the low and/or medium molecular weight PE preferably consists of low and/or medium molecular weight PE. According to a further version of the invention, the low and/or medium molecular weight PE consists of a mixture of low and of medium molecular weight PE or consists of low molecular weight PE alone or of medium molecular weight PE alone.

According to the invention, therefore, the presence of low and/or medium molecular weight PE is due to the need of reduce the viscosity of UHMWPE, the latter being—in at least one version of the invention—the preferred plastic material which constitutes the material and/or the resulting device. The mixture of UHMWPE and low and/or medium molecular weight PE therefore achieves such aim, namely, to allow the mouldability and extrudability of UHMWPE. Thanks to the present invention, therefore, such mixture is able to be extruded in a thread. In fact, according to an aspect of the present invention, by adding the low and/or medium molecular weight PE to the UHMWPE (or vice versa, namely, adding the UHMWPE to the low and/or medium molecular weight PE), it is possible to reduce the overall molecular weight of the mixture (and therefore of the UHMWPE) and therefore to reduce its viscosity, in order for such mixture to become extruded, for example, by a very powerful press. The obtained thread can be used as such for being feed to a 3D printing machine. Also, such obtained thread can be crushed into granules, the latter being used in an injection press.

It is advantageous, therefore, that for being able to extrude the UHMWPE from a press through thermal transformation (3D printing or press), the presence of a low and/or medium molecular weight PE is mandatory.

Considering LDPE and HDPE, when considered in the present invention, they have a molecular weight which is lower than the molecular weight of UHMWPE, and specifically they have low and/or medium molecular weight.

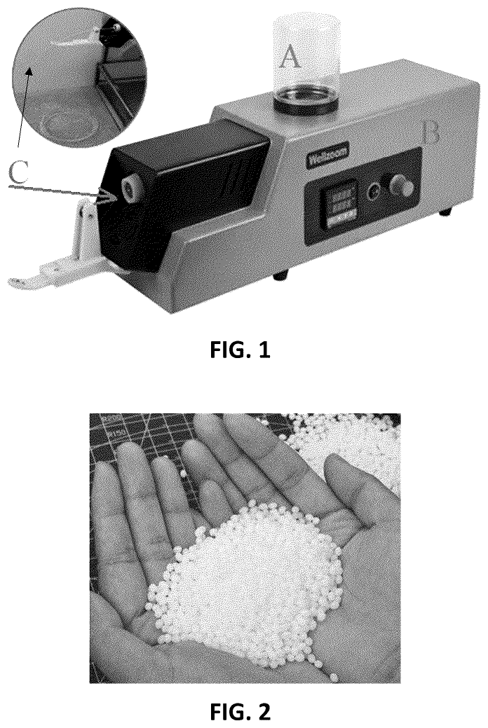

As shown in FIG. 1, an exemplary thread or filament extruder machine (or melter) B has a hopper A and the obtained thread is indicated with C. The pellets or granules of the plastic material or mixture are shown in FIG. 2, possibly/optionally to which is added at least one pharmaceutical or medical substances and/or a radiopacifying agent and/or a further additive. The pellets or granules are inserted into the hopper A of the melter B. In the melter B, there are heating means that at least partially melt the material inserted through the hopper A and the resulting filament C is extruded and obtained.

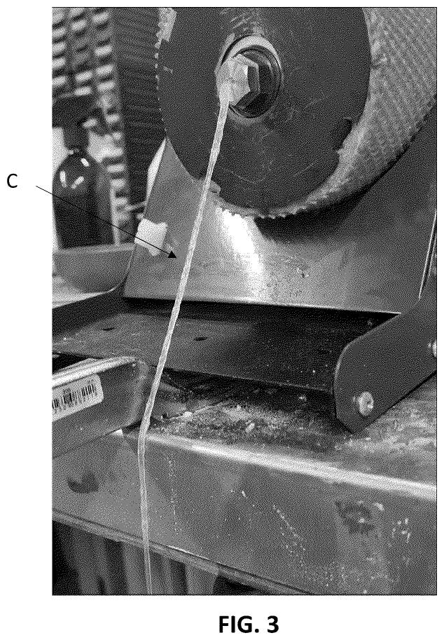

FIG. 3 depicts an exemplary enlarged view of a thread C extruded by an extruder according to an aspect of the present invention.

Such thread C is used as a feeding material of a 3D printer or it is crushed in (further) granules or pellet form, suitable to be moulded (injection or extrusion moulding).

Thanks to the low melting temperature of UHMWPE and the presence of the low and/or medium molecular weight PE (which reduces the viscosity of UHMWPE), such material can contain antibiotics, in order then to be moulded for example for obtaining joint spacer devices which require high mechanical performances.

Other spacer devices or devices in general can be obtained through 3D printing. In general, devices obtained in this way do not show strong mechanical performances and therefore can be used in locations where the loads are not too high (such as for example cranial prosthesis or cranial spacer devices o devices for example implantable in the shoulder).

At the end, it has been proved that the addition of at least one lower molecular weight PE to a UHMWPE is able to reduce the melting temperature of the latter and to drastically improve its flowability, being able to obtain a thread of a certain length, such as for example suitable to be wounded in an industrial coil. The length of the coil can reach 100 meters or some hundreds of meters. In any case, according to other versions of the invention, the extruded thread can have the length of approximately 1 meter (possibly shaped like a bar).

Otherwise, without the addition of lower molecular weight PE, UHMWPE can be extruded in an extremely difficult manner, and the resulting extruded portion is very short and small in size, or it is not extrudable or injection mouldable at all.

Usually, UHMWPE is obtained by static press into plates or bars and then it is transformed by tool machining (and not by moulding).

According to another embodiment of the invention, if the material has not to contain a pharmaceutical or medical substance which is sensitive to thermal degradation, such as for example at least one antibiotic, during the production steps, for example because the material has another purpose than infection healing, or because it incorporates such thermal sensitive substances after its production, it is possible to provide the material including also other plastic or resin materials, other than UHMWPE.

It is the case, for example, of a material which function is that to increase the bone integration (bio-integration) and/or ingrowth.

In such a case, the plastic or resin material can be added with at least one ceramic additive such for example a ceramic additive based on calcium, such as for example TCP (tricalcium phosphate) and/or HA (hydroxyapatite). Such ceramic additive, in fact, when present at least at the bone interface, has high ability to integration with the bone tissue surrounding the implant site and at the same time, without causing adverse reactions.

Therefore, the present material can comprise such ceramic additive (together with any possible ceramic material suitable to the purpose, such as for example the other ceramic material mentioned above), for example in powder form, can be added to plastic material such as for example of the insoluble type, such as for example PE, PMMA, PEEK, or to soluble-type plastic material, such as for example polylactic or polyglycolic acid polymers or other suitable polymers, such as polylactic and polyglycolic acid copolymers, polylactone, etc.

After, the mix of such ceramic additive with the selected plastic material, it is possible to melt such mixture, extrude the melted mixture in a thread to be used for a 3D printing machine or a thread which is crushed and used to fill an injection mould, in order to be subject to injection moulding and obtain the desired device. In such embodiment, the melting and/or transforming temperatures are high, even up or more than 300° ° C., but the ceramic additive is inorganic and therefore it is not subjected to degradation.

It is clear that, the higher the content of ceramic material, the higher is the integration of the bone tissue, but the resulting thread or device will be more fragile. On the contrary, the higher the content of plastic material, the higher the mechanical properties are in the resulting thread or device.

According to an example, which can be varied depending on the kind of resulting device to be obtained, the composition can comprise or consist of:
1) Plastic material (such as for example PE, PMMA, PEEK, PL, PG, etc. or mixture thereof): 30-80%,
2) Ceramic material or ceramic additive: 20-70%,
3) Radiopacifying agent: 0-15% or 5-15%.

In the present specification, the word thread is used a synonym of filament. However, at least according to a version of the invention, the word thread or filament is not synonym of fibre or sutures.

It should be noted that the possible additives and the pharmaceutical or medical substance present in the thread are homogeneously distributed in the whole volume of the thread, as they generally result from a mixing step.

According to another embodiment of the invention, the plastic material is PEEK. As PEEK melts at a high temperature, (about 350° C.), it is not possible for it to incorporate an antibiotics and be molded or extruded (the antibiotic would be degraded at such high temperature). Therefore, it is possible to obtain a thread of PEEK incorporating an antiseptic agent of inorganic nature, a bacteriostatic agent such as silver in its forms, such as metallic powder or salts comprising citrate, proteinate, colloidal, electrolytic, or other forms, or copper or gold in their forms or alloys, or as salts, boric acid. Preferably PEEK will be added with silver.

Furthermore, the PEEK can comprise a radiopacifying agent comprising metallic powders or heavy metals powers, of tungsten, tantalum, silver metallic powders or a metal salt of barium sulphate, zirconium oxide, bismuth oxide, etc.

At the same time, in combination or as an alternative, the PEEK can further comprise a ceramic material of the type indicated above, in order to increase its biocompatibility.

Using the resulting thread in 3D printing or crushing it and using the resulting crushed or granulated material in a moulding technique, the resulting implant will be more biocompatible and the fibrous sheath (originating after the implant) will be thinner than usual.

The invention thus conceived can undergo numerous modifications and variants all covered by the inventive concept.

The characteristics presented for one version or embodiment can be combined with the characteristics of another version or embodiment, without departing from the scope of protection of the present invention.

Moreover, all of the details can be replaced by other technically equivalent elements. In practice, the materials used, as well as the contingent shapes and sizes, can be whatever according to requirements without for this reason departing from the scope of protection of the following claims.

1. A method for the obtainment of a thread made of a material biocompatible and implantable in the human body, comprising the following steps:
providing UHMWPE as a plastic base material, wherein said UHMWPE has a molecular weight and is in granular or pellet form,
providing a polyethylene material with a molecular weight which is lower than the molecular weight of the UHMWPE, wherein said polyethylene material is in powder, granular or pellet form,
adding said polyethylene material to the UHMWPE or vice versa,
mixing said polyethylene material with the UHMWPE, obtaining a plastic mixture,
providing a pharmaceutical or medical substance comprising at least one antibiotic,
adding said pharmaceutical or medical substance to said plastic mixture,
mixing said pharmaceutical or medical substance with said plastic mixture, obtaining an added base mixture, wherein said pharmaceutical or medical substance is uniformly distributed in the added base mixture,
heating said added base mixture in an extruder at a predetermined temperature which is lower than the degradation temperature of said pharmaceutical or medical substance, obtaining a melted material,
extruding said melted material in order to obtain said thread made of said heated material,
cooling down said thread, and
moulding said thread in order to obtain a device able to be implanted in the human body such as a catheter, screw or joint, coupling or mesh, spacer device, prosthesis or prosthetic device.

2. The method according to claim 1, wherein said polyethylene material comprises or is a low molecular weight polyethylene, a medium molecular weight polyethylene, a low density polyethylene, a high density polyethylene or a mixture thereof.

3. The method according to claim 1, wherein said step of adding comprises adding in incremental steps said polyethylene material to the UHMWPE or vice versa, and said step of mixing comprises mixing said polyethylene material with the UHMWPE after each incremental step of adding said polyethylene material.

4. The method according to claim 1, wherein said moulding step of said thread comprises the following steps:
feeding said thread to a tridimensional printer and moulding said device able to be implanted in the human body through said tridimensional printer.

5. The method according to claim 1, therein said moulding step of said thread comprises the following steps:
crushing or granulating said thread in order to obtain a crushed or granulated material,
placing said crushed or granulated material in a mould, in order to perform an injection moulding step or a compression moulding step and obtain a device able to be implanted in the human body,
wherein said injection moulding step or said compression moulding step occurs at a predetermined temperature which is lower than the degradation temperature of said pharmaceutical or medical substance.

6. The method according to claim 1, wherein said moulding through said tridimensional printer occurs at a predetermined temperature which is lower than the degradation temperature of said pharmaceutical or medical substance, for example equal or less than 160° C.

7. The method according to claim 1, wherein said step of providing a plastic mixture comprises providing:
UHMWPE and low molecular weight PE, or
UHMWPE and medium molecular weight PE, or
UHMWPE, low molecular weight PE and medium molecular weight PE, or
UHMWPE and low density PE having a molecular weight lower than the molecular weight of UHMWPE, or
UHMPE and high density PE having a molecular weight lower than the molecular weight of UHMWPE, or
UHMWPE and low density PE and high density PE, both having a molecular weight lower than the molecular weight of UHMWPE, or
UHMWPE and high density PE, wherein said high density PE is a linear polymer and/or wherein said high density PE has a low and/or medium molecular weight, and/or
wherein the low molecular weight PE has a molecular weight in the range 200.000 to 500.000, and/or
wherein the medium molecular weight PE has a molecular weight in the range 500.000 to 1 million.

8. The method according to claim 1, wherein said plastic mixture comprises UHMWPE in one of the following percentages: at least 40%.

9. The method according to claim 1, wherein said material comprises:
low and/or medium molecular weight PE or low density PE and/or high density PE:
from 5% to 40% or from 5% to 20%;
a pharmaceutical or medical substance comprising gentamicin sulphate from 5% to 10%;
another pharmaceutical or medical substance, comprising vancomycin from 0% to 10% or from 5% to 10%,
a radiopacifying agent: from 0% to 20% or from 10% to 20%; and
UHMWPE: up to 100%.

10. The method according to claim 1, comprising the following steps:
providing and mixing with said plastic mixture a further additive comprising a soluble and/or reabsorbable ceramic material, in the form of powder or granules, comprising tricalcium phosphate or calcium sulphate or hydroxyapatite or a mixture thereof, and/or at least a coloring substance of the biocompatible type and adapted to be introduced in the human body, and/or
providing and mixing said at least one antibiotic comprising or consisting of at least one of gentamicin sulphate, Amikacin, Azlocillin, Aztreonam, Clarithromycin, Chloramphenicol, Ciprofloxacin, Clindamycin, Coumermycin, Fosfomycin, Josamycin, Kanamycin, Mezlocillin, Mupirocin, Nalidixic acid, Netilmicin, Norfloxacin, Novobiocin, Ofloxacin, Oxacillin, Sulbactam, Tobramycin, Trimethoprim, Trimethoprim together with Sulphamethoxazole, Vancomycin, another suitable antibiotic which is thermoresistant and that is active against microbial infections, etc., and/or
providing and mixing a pharmaceutical or medical substance further comprising an antiseptic agent of organic or inorganic nature, a bacteriostatic agent such as silver in its forms, such as metallic powder or salts comprising citrate, proteinate, colloidal, electrolytic, or other forms, or copper or gold in their forms or alloys, or as salts, boric acid, and/or
providing a radiopacifying agent comprising metallic powders or heavy metals powders, of tungsten, tantalum, silver metallic powders or a metal salt of barium sulphate, zirconium oxide, bismuth oxide.

11. The method according to claim 1, wherein said step of obtaining a plastic mixture and said heating step occur simultaneously in said extruder.

12. The method according to claim 1, wherein said step of adding said further polyethylene to the UHMWPE occurs step-by-step, adding said further polyethylene a little at a time to said UHMWPE or wherein said step of adding said further polyethylene to the UHMWPE occurs adding in one run all the predetermined quantity of further polyethylene to said UHMWPE, or
wherein said step of adding said further polyethylene to the UHMWPE occurs step-by-step, adding UHMWPE to said further polyethylene a little at a time or wherein said step of adding said further polyethylene to the UHMWPE occurs adding in one run all the predetermined quantity of UHMWPE to said further polyethylene.

13. A method for the obtainment of a thread made of a material biocompatible and implantable in the human body, comprising the following steps:
providing a plastic material comprising an acrylic resin or polymethylmethacrylate (PMMA) or polyethylene (PE) or low density polyethylene or high density polyethylene or ultra high molecular weight polyethylene (UHMWPE) or polypropylene or polyamide or polyetheretherketone (PEEK) or polylacic acid or polyglycolic acid polymer or copolymer or a mixture thereof, wherein said plastic material is in powder, granular or pellet form,
providing a ceramic additive comprising a soluble and/or reabsorbable ceramic material, in the form of powder or granules, comprising tricalcium phosphate or calcium sulphate or hydroxyapatite or a mixture thereof,
adding and mixing said ceramic additive with said plastic material, obtaining an added material, wherein said ceramic additive is uniformly distributed in said mixture,
heating said added material in an extruder at a predetermined temperature, obtaining a melted material,
extruding said melted material in order to obtain said thread made of said heated material, and
cooling down said thread.

14. The method according to claim 13, comprising the following steps:
providing and mixing at least a coloring substance of the biocompatible type and adapted to be introduced in the human body, and/or
providing and mixing a pharmaceutical or medical substance comprising at least one antibiotic comprising at least one of gentamicin sulphate, Amikacin, Azlocillin, Aztreonam, Clarithromycin, Chloramphenicol, Ciprofloxacin, Clindamycin, Coumermycin, Fosfomycin, Josamycin, Kanamycin, Mezlocillin, Mupirocin, Nalidixic acid, Netilmicin, Norfloxacin, Novobiocin, Ofloxacin, Oxacillin, Sulbactam, Tobramycin, Trimethoprim, Trimethoprim together with Sulphamethoxazole, Vancomycin, and/or
providing and mixing a pharmaceutical or medical substance comprising an antiseptic agent of organic or inorganic nature, a bacteriostatic agent such as silver in its forms, such as metallic powder or salts comprising citrate, proteinate, colloidal, electrolytic, or other forms, or copper or gold in their forms or alloys, or as salts, boric acid, and/or
providing a radiopacifying agent comprising metallic powders or heavy metal powders, of tungsten, tantalum, silver metallic powders or a metal salt of barium sulphate, zirconium oxide, bismuth oxide.

15. The method according to claim 13, comprising the following steps:
crushing or granulating said thread in order to obtain a crushed or granulated material,
placing crushed or granulated material in a mould, in order to perform an injection moulding step or a compression moulding step,
wherein said injection moulding step or a compression moulding step.

16. The method according to claim 13, comprising the following steps:
feeding said thread to a 3D printer,
moulding a device able to be implanted in the human body through said 3D printer.

17. The method according to claim 13, wherein said mixing or adding step and said heating step of said material occur simultaneously in said extruder.

18. The method according to claim 13, wherein said added material comprises:
plastic material: 30-80%,
further ceramic additive: 20-70%, and
radiopacifying agent: 0-15% or 5-15%.

19. The method according to claim 1, wherein said material comprises:
a plastic material comprising:
UHMWPE and low molecular weight PE, or
UHMWPE and medium molecular weight PE, or
UHMWPE, low molecular weight PE and medium molecular weight PE, or
UHMWPE and low density PE having a molecular weight lower than the molecular weight of UHMWPE, or
UHMPE and high density PE having a molecular weight lower than the molecular weight of UHMWPE, or
UHMWPE and low density PE and high density PE, both having a molecular weight lower than the molecular weight of UHMWPE,
said further ceramic additive,
said radiopacifying agent, and
said at least one pharmaceutical or medical substance, or
wherein said material comprises:
a plastic material that is PEEK,
said further ceramic additive,
said at least one pharmaceutical or medical substance that is a bacteriostatic agent, and said radiopacifying agent.

20. A method for the obtainment of a device implantable in the human body, wherein said device is a temporary and/or disposable prosthetic device or spacer device for treating a bone or a joint location or a catheter, screw or joint, coupling or mesh, comprising the following steps:
providing said thread made of the material biocompatible and implantable in the human body obtained with the method of claim 1,
molding said at least one thread by means of a tridimensional printer in order to obtain said device, or
crushing or granulating said thread in order to obtain a crushed or granulated material, providing said crushed or granulated material in a mould,
moulding said crushed or granulated material by means of injection molding or by means of compression press, in order to obtain said device,
providing a radiopacifying agent to said thread or to said device before said moulding step,
providing a pharmaceutical or medical substance, if desired comprising at least one antibiotic, to said material or to said device before or after said moulding step.

21. The method according to claim 20, wherein said tridimensional printing step occurs at a temperature equal to or less than 160° C.
